(19) United States
(12) Patent Application Publication
(10) Pub. No.: US 2026/0283584 A1
(43) Pub. Date: Sep. 24, 2026

(54) DIAGNOSTIC DEVICE FOR EXERCISE INDUCED LARYNGEAL OBSTRUCTION (EILO) AND USES THEREOF

(71) Applicant: CHILDREN'S NATIONAL MEDICAL CENTER, Washington, DC (US)

(72) Inventors: Nancy BAUMAN, Washington, DC (US); Kevin CLEARY, Washington, DC (US); Trevor JEHL, Washington, DC (US); Pamela MUDD, Washington, DC (US); Mahadin AHMED, Washington, DC (US); Renae BITOR, Washington, DC (US); Rosy JAMES, Washington, DC (US); Mai Phuong LE, Washington, DC (US)

(73) Assignee: CHILDREN'S NATIONAL MEDICAL CENTER, Washington, DC (US)

(21) Appl. No.: 19/471,504

(22) PCT Filed: Apr. 5, 2024
(86) PCT No.: PCT/US2024/023414

§ 371 Date: Oct. 1, 2025

Related U.S. Application Data
(60) Provisional application No. 63/494,914, filed on Apr. 7, 2023.
(60) Provisional application No. 63/499,383, filed on May 1, 2023.

(51) Int. Cl. A61B 7/00; A61B 5/00; A61B 5/085; A61B 7/04; A63B 24/00

(52) U.S. Cl. CPC A61B 7/003; A61B 5/0004; A61B 5/085; A61B 5/6822; A61B 5/6823; A61B 5/6831; A61B 7/04; A63B 24/0062; A61B 2562/0261

(57) ABSTRACT

A wearable apparatus, including a sound sensor configured to acquire an audio signal from a throat of a wearer; a strain gauge coupled to a flexible band and configured to measure a strain of the strain gauge when the flexible band is in contact with a chest of the wearer during a respiratory cycle of the wearer and output a respiratory signal corresponding to the strain; and processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry.

### CROSS-REFERENCE TO RELATED APPLICATIONS

The present application claims priority to U.S. Provisional Application No. 63/494,914, filed Apr. 7, 2023, and to U.S. Provisional Application No. 63/499,383, filed May 1, 2023, each of which is incorporated herein by reference in its entirety for all purposes.

### FEDERAL FUNDING DISCLOSURE

This disclosure was made with government support under Grant Number EB029381 awarded by the National Institutes of Health. The government has certain rights to the disclosure.

### BACKGROUND

### Field of the Disclosure

The present disclosure relates to detection of exercise-induced laryngeal obstruction.

### Description of the Related Art

Exercise-induced laryngeal obstruction (EILO) refers to the dynamic narrowing of the larynx that restricts inhalation and causes acute dyspnea. EILO may result from paradoxical vocal fold movement (PVFM)—where the vocal folds involuntarily adduct during inspiration—or from supraglottic collapse. Sometimes, these physiological phenomena occur concurrently in exercise states, although PVFM occurs far more commonly than supraglottic collapse.

Diagnosing EILO can be challenging because symptoms typically resolve shortly after the inciting activity stops. Without witnessing the acute event, symptoms are often mistakenly attributed to exercise-induced bronchospasm, asthma, or an allergic reaction by health care workers. Such misdiagnoses lead to the prescription of many ineffective medications for managing EILO, including bronchodilators, antihistamines, and steroids.

Physiologically, EILO is distinct from the bronchoconstriction of these other conditions. Patients with EILO have shortness of breath with inspiration, whereas those with asthma or bronchoconstriction have difficulty with expiration. Like bronchoconstriction, voluntary vocalization (talking) also only occurs on expiration. Recognizing this crucial distinction inspired us to design a mobile diagnostic device that can be worn by individuals with suspected EILO during their exercise of choice. Recording vocalization in relation to vocal fold activity allows detection of inspiratory stridor, diagnostic of EILO.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry, wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.

In one embodiment, the present disclosure is related to a wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry.

In one embodiment, the present disclosure is related to a wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing coupled to a wristband and configured to synchronize acquisition of the audio signal and acquisition of the respiratory signal, receive the audio signal from the sound sensor and the respiratory signal from the strain gauge, and transmit the audio signal and the respiratory signal to external processing circuitry, wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is a schematic of a sound sensor, according to one embodiment of the present disclosure;

FIG. 1B is a schematic of a sound sensor, according to one embodiment of the present disclosure;

FIG. 1C is a schematic of a sound sensor, according to one embodiment of the present disclosure;

FIG. 1D is a schematic of a sound sensor, according to one embodiment of the present disclosure;

FIG. 2A is a schematic of a strain gauge, according to one embodiment of the present disclosure;

FIG. 2B is a schematic of a strain gauge, according to one embodiment of the present disclosure;

FIG. 3 is a schematic of a microcontroller housing, according to one embodiment of the present disclosure;

FIG. 4 is an illustration of a wearable apparatus, according to one embodiment of the present disclosure;

FIG. 5 is an illustration of a wearable apparatus, according to one embodiment of the present disclosure;

FIG. 6 is an illustration of a wearable apparatus, according to one embodiment of the present disclosure;

FIG. 7 is an illustration of a wearable apparatus, according to one embodiment of the present disclosure;

FIG. 8 is an illustration of a wearable apparatus, according to one embodiment of the present disclosure;

FIG. 9 is a respiratory signal, according to one embodiment of the present disclosure;

FIG. 10 is a synchronized respiratory signal and audio signal, according to one embodiment of the present disclosure;

FIG. 11 is a schematic of a user device for performing a method, according to one embodiment of the present disclosure;

FIG. 12 is a schematic of a hardware system for performing a method, according to one embodiment of the present disclosure; and FIG. 13 is a schematic of a hardware configuration of a device for performing a method, according to one embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed to measurement of biological signals (biosignals) related to respiration in order to assess Exercise Induced Laryngeal Obstruction (EILO). EILO can result in inspiratory stridor, e.g., noisy inspiration resulting from irregular inflow of air during exercise. Most cases of EILO are due to inappropriate involuntary adduction (closure) of the vocal folds during inspiration, termed paradoxical vocal fold motion (PVFM), while a smaller percentage of cases are due to collapse of the supraglottic structures (exercise-induced laryngomalacia). Both conditions manifest with similar inspiratory stridor and dyspnea. PVFM is also known as paradoxical vocal cord dysfunction (PVCD), a form of vocal cord dysfunction (VCD).

Symptoms of EILO generally can include, but are not limited to, irregular airflow through the airway, difficulty breathing, tightness of the throat, noisy breathing (e.g., stridor, wheezing), coughing, etc. These symptoms can be similar to those of asthma or bronchoconstriction, resulting in difficulty distinguishing and diagnosing EILO in particular. Asthma and bronchoconstriction (e.g., exercise induced bronchoconstriction (EIB) are characterized by respiratory challenges during expiration, while EILO is related to inspiration. However, it can be difficult to determine whether inspiration or expiration is problematic based on symptoms alone without witnessing an episode of EILO. Furthermore, EILO can be triggered by exercise, physical exertion, or strenuous activity, and symptoms of EILO and underlying causes can subside when a patient is at rest or not performing strenuous activity. It can be difficult to collect biosignal measurements related to EILO in a physiology lab while inducing laryngeal obstruction via physical activity.

In one embodiment, the systems and methods described herein can be used to acquire biosignal measurements related to EILO when a subject (patient) is in motion, e.g., walking, running, exercising, etc. The biosignal measurements can be used to detect inspiratory stridor caused by vocal cord activity by determining whether vocal cord activity occurs and whether the vocal cord activity occurs during inspiration, as occurs with EILO. In one embodiment, the biosignal measurements can be used to provide a diagnosis of EILO.

In one embodiment, the present disclosure is directed to a wearable apparatus including at least one vocal cord activity sensor and at least one respiratory sensor. The vocal cord activity sensor can include a sound sensor, e.g., a microphone. The sound sensor can be configured to convert sound waves into electrical signals (audio signals) for signal processing. The at least one respiratory sensor can include a strain gauge configured to measure strain applied to the gauge. The strain gauge can be affixed to the chest of a subject (patient) and can convert strain, which is caused by movement of the chest, to electrical signals (respiratory signals) for signal processing. The movement of the chest can correspond to a respiratory cycle, including inhalation and expiration. In one embodiment, the apparatus can further include or be in communication with processing circuitry configured to synchronize the audio signals and the respiratory signals (collectively, biosignals) and process the biosignals to identify vocal cord activity, including vocal cord movement, during inspiration.

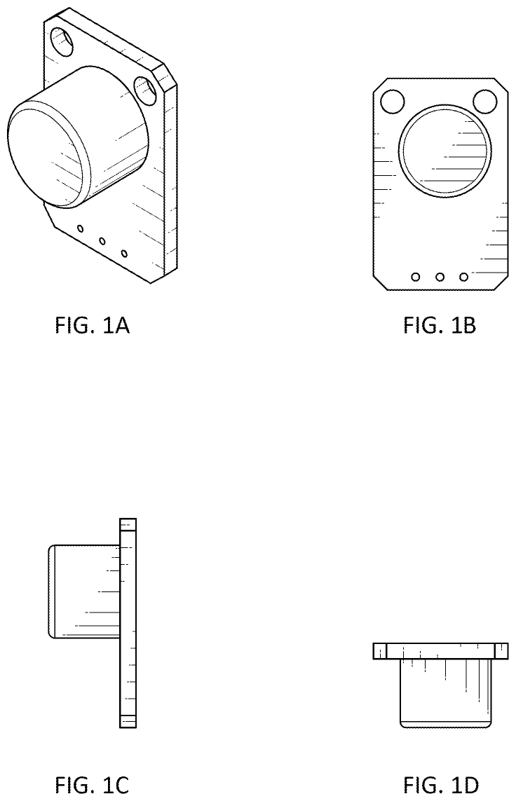

FIG. 1A through FIG. 1D are illustrations of a microphone used to acquire audio signals according to one embodiment. In one embodiment, the microphone can record and identify audio signals corresponding to vocal cord (or vocal fold) activity. In one example, audio signals corresponding to vocal cord activity can have a frequency greater than approximately 400-600 Hz. The audio signals corresponding to vocal cord activity can be involuntary sounds that are made by movement of the vocal cords themselves rather than active sound made by a wearer. In one embodiment, the microphone of the apparatus can be positioned at a throat of a wearer. The microphone can detect and record audio signals from the throat, where the vocal cords are located. The position of the microphone of the apparatus can include, but is not limited to, at one or both sides of the throat, along the center of the throat, under the chin or jawbone, etc. In one embodiment, the microphone can be covered by a housing for user comfort.

In one example, the microphone can be a MAX4466 microphone. In one embodiment, a gain can be applied to the audio signals acquired by the microphone by an amplifier. As an example, the amplifier can be an Arceli Electret Microphone Amplifier configured to acquire audio signals within a frequency range of approximately 20 Hz to approximately 20 kHz with a sensitivity of −44+/−2 decibels (dB). In one embodiment, the sound sensor can include audio signal processing circuitry and/or memory configured to receive, store, process, and transmit the audio signals. The sound sensor can be verified by acquiring an audio signal of a tone of predetermined frequency and verifying that a frequency domain representation of the acquired audio signal includes a peak at the predetermined frequency.

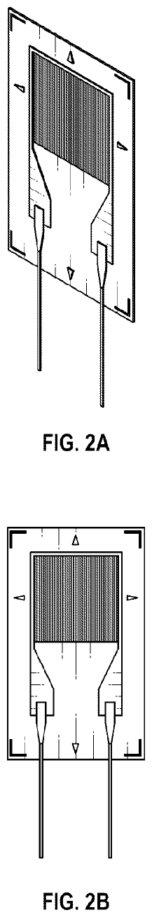

FIG. 2A and FIG. 2B are illustrations of a strain gauge (or strain sensor) used to acquire respiratory signals. In one embodiment, the strain gauge can be directly taped to the body. In one embodiment, the strain gauge can be located at the upper abdomen where the anterior serratus muscles are located. In one embodiment, the strain gauge can be contained in a housing. In one embodiment, a strain gauge can be coupled to (e.g., affixed to) a flexible band. The flexible band can be attached to the chest of a wearer. In one example, the flexible band can be attached at the ribs of the wearer or the chest of the wearer. The flexible band can be a horizontal band spanning across at least a portion of the width of the torso of the wearer. When the wearer inhales and exhales, the movement of the chest causes the flexible band to bend. The electrical resistance of the strain gauge is affected by the degree and direction of bend of the strain gauge, resulting in different voltage drops across the strain gauge (measured as the respiratory signal) when the bend changes with movement of the chest. The change in voltage of the respiratory signal can therefore be used to determine a degree and direction of bend of the strain gauge.

In one example, the strain gauge can be a BF350-3AA/1.5AA strain gauge. A signal output of the strain gauge can be amplified by a Taidacent Strain Gauge 350 Ohm Bend Flex Sensor Weighing Amplifier Module having a sensitivity factor of 2, strain limit of 2%, and maximum resistance of approximately 350 Ohms. In one embodiment, a clockwise bend of the strain gauge can result in an increase in the respiratory signal, while a counterclockwise bend of the strain gauge can result in a decrease in the respiratory signal. The decrease in the respiratory signal can include negative values of the respiratory signal. A clockwise bend of the strain gauge can correspond to chest expansion, e.g., as a result of inhalation. A counterclockwise bend of the strain gauge can correspond to chest compression, e.g., as a result of exhalation. In one embodiment, the strain gauge can be connected to respiratory signal processing circuitry and/or memory configured to receive, store, process, and transmit the respiratory signals. In one embodiment, a respiratory signal can be characterized by a frequency corresponding to a rate of breathing and/or an amplitude corresponding to a maximum or minimum chest movement during breathing.

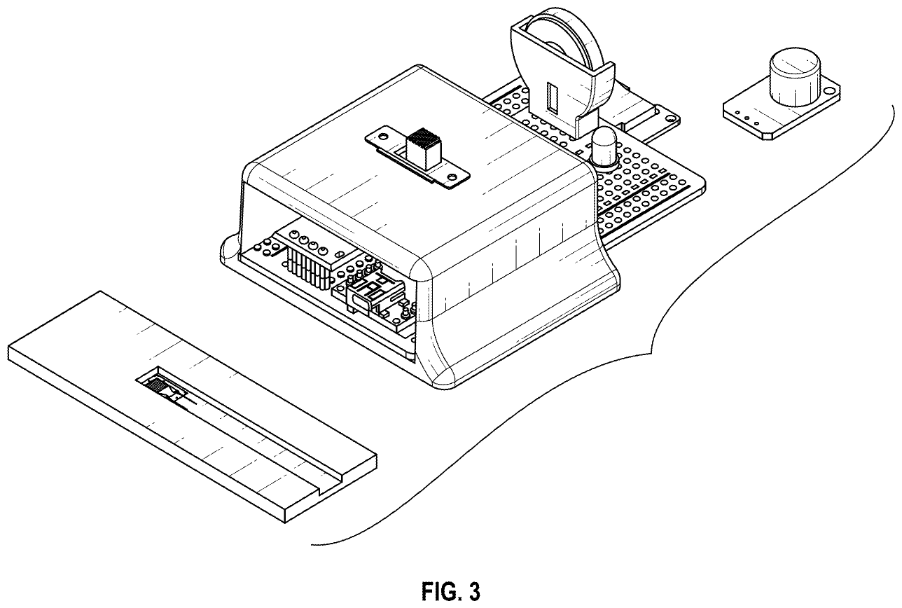

FIG. 3 is an illustration of processing circuitry according to one embodiment of the present disclosure. In one embodiment, the processing circuitry can include a microcontroller (e.g., an Arduino Nano RP2040 microcontroller). The microcontroller can control the sensors (e.g., sound sensor, respiratory sensor) and can receive biosignals from the sensors via a wired connection or a wireless connection. In one embodiment, the biosignals can be amplified by an amplifier module. The amplifier module can be electronically coupled to a sensor (e.g., the sound sensor or the strain gauge) or to the microcontroller. In one embodiment, the microcontroller can transmit instructions to one or more of the sensors. The instructions can include, for example, a sampling rate, a sampling period, transmission settings, etc. For example, the instructions can include a repeatable protocol for signal acquisition and transmission.

In one embodiment, the microcontroller can be coupled to memory, e.g., one or more secure digital (SD) cards. The microcontroller can store biosignals in the memory. In one embodiment, the microcontroller can be configured to transmit data (e.g., the biosignals) to a second processing device. The second processing device can be, for example, a user device, a computer, a server, a mobile device, etc. In one embodiment, the microcontroller can be configured for wired or wireless communication with the second processing device. In one embodiment, the memory can be uncoupled from the microcontroller in order to transfer data stored in the memory to the second processing device.

In one embodiment, the microcontroller and related hardware (e.g., a battery, memory, wireless transmission modules) can be contained in a housing, as illustrated in FIG. 3. In one embodiment, the batteries can supply 12V power to the microcontroller for consistent and repeated signal acquisition and sensor control. In one embodiment, the housing can include a power switch to turn on the microcontroller and/or the sensors. In one embodiment, the housing can include a signal acquisition switch. Activation of the signal acquisition switch can result in the microcontroller transmitting an acquisition initialization signal to at least one of the sensors (e.g., the strain gauge, the sound sensor). The at least one sensor can begin acquiring signal data when the acquisition initialization signal from the microcontroller is received. In one embodiment, the housing can be a flexible material, such as a resin. In one embodiment, the housing can be attached to the body, as will be described herein. In one embodiment, the housing can be positioned separately from the body and the microcontroller can transmit and receive signals via a wireless communication network, e.g., Bluetooth, WiFi. In one embodiment, the microcontroller can communicate with sensors (e.g., the strain gauge, the sound sensor) via a wireless connection or wireless communication network, e.g., Bluetooth, WiFi. For example, the microcontroller can be paired with a microphone and a strain gauge over a Bluetooth connection. The microphone can transmit audio signals to the microcontroller wirelessly, and the strain gauge can transmit respiratory signals to the microcontroller wirelessly. Similarly, the microcontroller can transmit instructions to the sensors wirelessly.

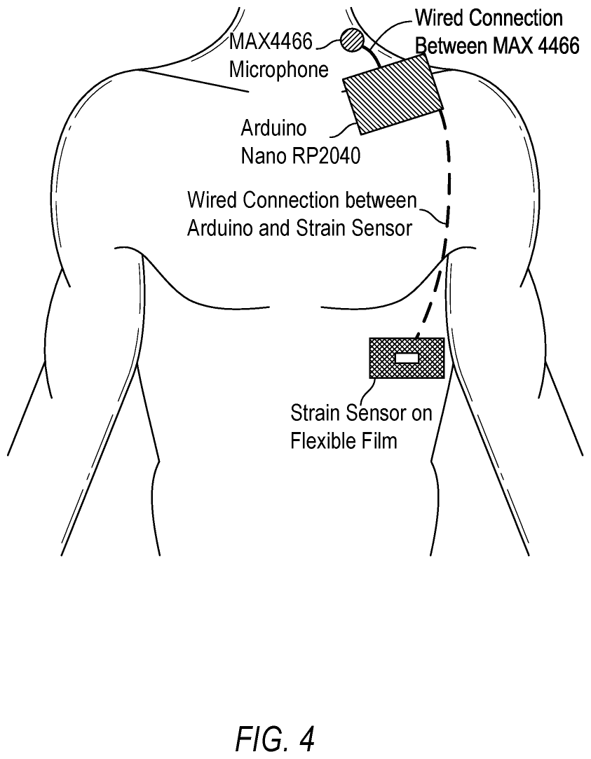

FIG. 4 is an illustration of the wearable apparatus according to one embodiment of the present disclosure. In one embodiment, the sound sensor and the strain gauge can be communicatively connected to a microcontroller (e.g., an Arduino Nano RP2040 microcontroller) via a wireless connection or a wired connection, as illustrated in FIG. 4. In one embodiment, the microcontroller illustrated in FIG. 4 can be contained in a housing, as described with reference to FIG. 3. The sound sensor can be positioned at the throat and the strain gauge can be positioned at the ribs of the wearer. In one embodiment, the sensors and microcontroller can be affixed to the torso of the wearer with a medical adhesive. In one embodiment, the sensors and microcontroller can be affixed to the wearer's shirt. For example, the sensors and the microcontroller can be coupled to clips, and each clip can be attached to the wearer's shirt.

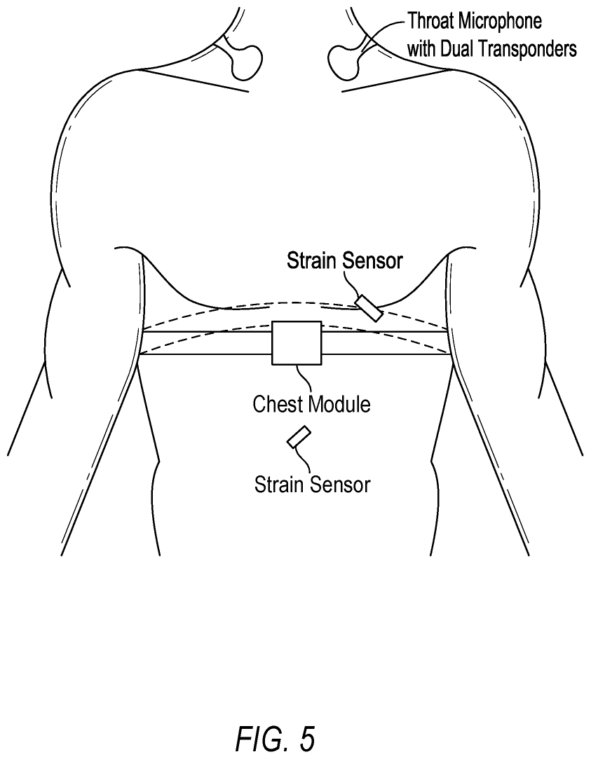

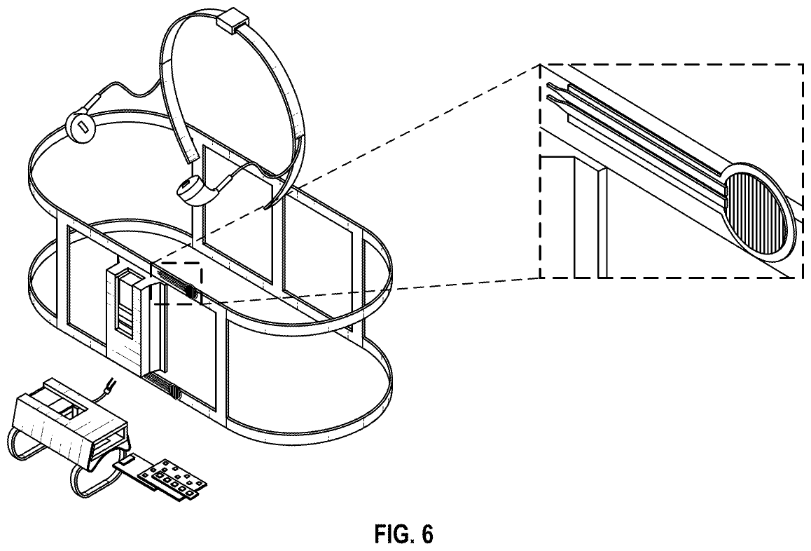

FIG. 5 is an illustration of the wearable apparatus according to one embodiment of the present disclosure. In one embodiment, the sound sensor can include a dual throat microphone having a first wireless transponder and a second wireless transponder. Each transponder can be positioned on one side of the throat. In one embodiment, the wireless transponders can transmit audio signals wirelessly, e.g., over a Bluetooth connection, wireless network, etc. to a microcontroller. The respiratory sensor can include a first strain sensor and a second strain sensor. The first strain sensor can be positioned at the ribs of the wearer and the second strain sensor can be positioned at the diaphragm of the wearer. In one embodiment, a strain sensor can be affixed to a flexible band around the torso of the wearer. In one embodiment, the strain sensors can transmit respiratory signals wirelessly, e.g., over a Bluetooth connection, wireless network, etc. to a microcontroller. In one embodiment, the wearable apparatus can include a chest module. The chest module can include processing circuitry (e.g., the microcontroller of FIG. 3 or respiratory signal processing circuitry) and can receive signals from the strain sensors. The chest module can be contained in a housing and affixed to the flexible band. In one embodiment, the chest module can be in communication with the microcontroller of FIG. 3. The microcontroller can be located separately from the body.

FIG. 6 is an illustration of the wearable apparatus according to one embodiment of the present disclosure. The sensors and the chest module can be coupled to a flexible harness. The coupling mechanism can be a clip or an adhesive. In one embodiment, the sensors can be fitted into an opening in the harness. The harness can include a neckpiece. The neckpiece can be an open or closed loop that can be worn around the neck. A microphone can be coupled to the neckpiece so that the microphone is positioned at the throat when the neckpiece is worn around the neck. In one embodiment, a microphone can be attached to an end of the neckpiece via a wire. In one embodiment, the diameter of the neckpiece can be adjustable. The harness can further include a chest strap. The chest strap can be a flexible or semi-rigid material that can be worn around the torso. In one embodiment, the respiratory sensor can be a force sensor coupled to the chest strap. The resistance of a force sensor can vary based on a force applied to the force sensor. The movement of the chest can result in a force on the force sensor. The force can be applied by the flexing of the chest strap when the chest expands and contracts. The resulting signal can be a respiratory signal. In one embodiment, the force sensor can be coupled to the chest strap so that the force sensor is positioned on the front of the chest when the chest strap is worn around the torso. In one embodiment, respiratory signal processing circuitry associated with the force sensor (e.g., a chest module) can also be coupled to the chest strap.

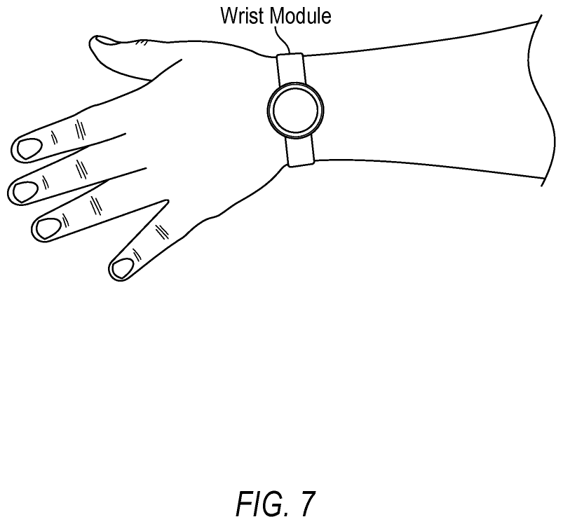

In one embodiment, processing circuitry, e.g., the microcontroller of FIG. 3, can be coupled to a wrist cuff as a wrist module. FIG. 7 is an illustration of a wrist module coupled to a wrist cuff, according to one embodiment of the present disclosure. In one embodiment, the sensors on the harness can transmit biosignals to the microcontroller on the wrist via a wireless connection. In this manner, the microcontroller can be kept in close proximity to the sensors on the harness without needing to be attached to the torso of the wearer. Wearing the microcontroller on a wrist cuff can reduce the risk of the microcontroller becoming uncoupled and dislodged when the body is in motion, e.g., running.

In one embodiment, a sensor can be coupled to a wrist cuff as a wrist module. For example, a microphone can be coupled to the wrist cuff. In one example, the microphone can be contained in a housing of the wrist cuff. The microphone can be in wireless communication with a microcontroller. Placement of the microphone on the wrist cuff can provide flexibility for when and where the microphone is positioned to record audio signals. For example, a wearer can hold their wrist up to their throat when they are experiencing symptoms of EILO and want to record audio. In one embodiment, the wearable apparatus can include a left wrist cuff and a right wrist cuff. Sound sensors can be coupled to both or either of the left wrist cuff and the right wrist cuff.

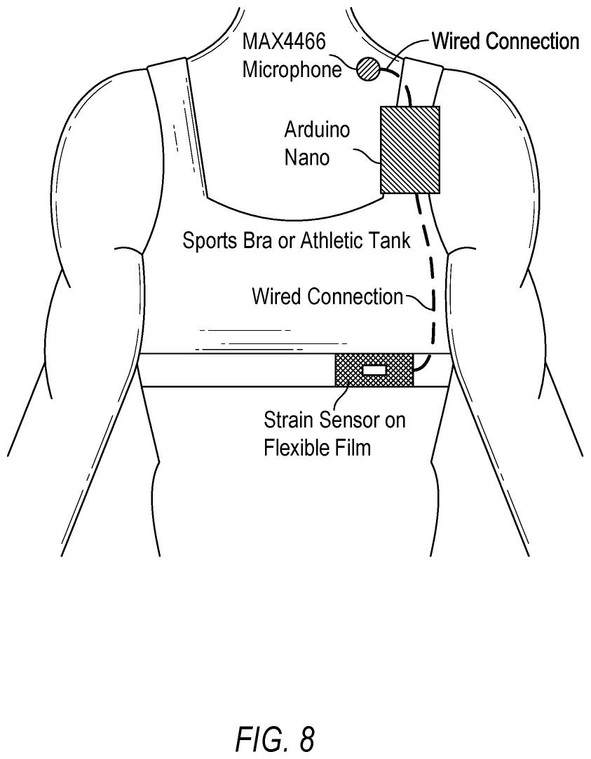

FIG. 8 is an illustration of the wearable apparatus coupled to an article of clothing according to one embodiment of the present disclosure. The article can be, for example, a bra top, tank top, shirt, vest, etc. In one embodiment, the article can comprise one or more flexible (e.g., fabric) bands or straps that can be worn on any part of the body. In one embodiment, the sensors and a microcontroller can be attached to the article via a clip or an adhesive. In one embodiment, the sensors can be sewn to the article. For example, the strain gauge can be sewn into a chest band of the article. In one embodiment, the sensors can be connected to the microcontroller via a wired connection, as illustrated in FIG. 8. The wired connection can be sewn into or attached to the article to prevent movement when the wearer is in motion.

It can be appreciated that the configurations described and illustrated herein are presented as non-limiting embodiments, and that the sensors (e.g., microphones, strain gauges) and processing circuitry can be affixed at or near the body of a wearer in a variety of ways. For example, the sensors can be affixed to the skin via a medical adhesive. In one embodiment, the sensors can be coupled to clips, adhesive, or hook-and-loop fabric (e.g., Velcro) that can be removably attached to a wearer's clothing. Similarly, the processing circuitry (e.g., the microcontroller) can be attached to a wearer's body or apparel, as described herein. For example, the microcontroller can be contained in a housing, and the housing can be clipped to a wearer's shirt collar. The processing circuitry can be coupled to the sensors via a wired connection when the processing circuitry is positioned on or near the wearer's body. In one embodiment, the sensors and the processing circuitry can be configured for wireless communication, and the processing circuitry need not be worn on the body.

In one embodiment, the biosignals from the sensors can be processed to identify vocal cord activity during inspiration. The biosignals can be processed by the microcontroller and/or a user device. In one embodiment, the microcontroller can receive the biosignals from the sensors and can transmit the biosignals to a user device for signal processing. In one embodiment, a user device can pre-process the biosignals. Pre-processing can include, but is not limited to, filtering, smoothing, noise reduction, time-shifting, normalization, transformation to a frequency domain, etc. in order to generate a clean biosignal.

The respiratory signals can indicate the rise and fall of the chest associated with inhalation and exhalation in the respiratory cycle. The respiratory signals can be approximately periodic. The rise (expansion) of the chest associated with inhalation can result in an increase in the amplitude of the respiratory signal, while the fall (deflation) of the chest associated with exhalation can result in a decrease in the amplitude of the respiratory signal.

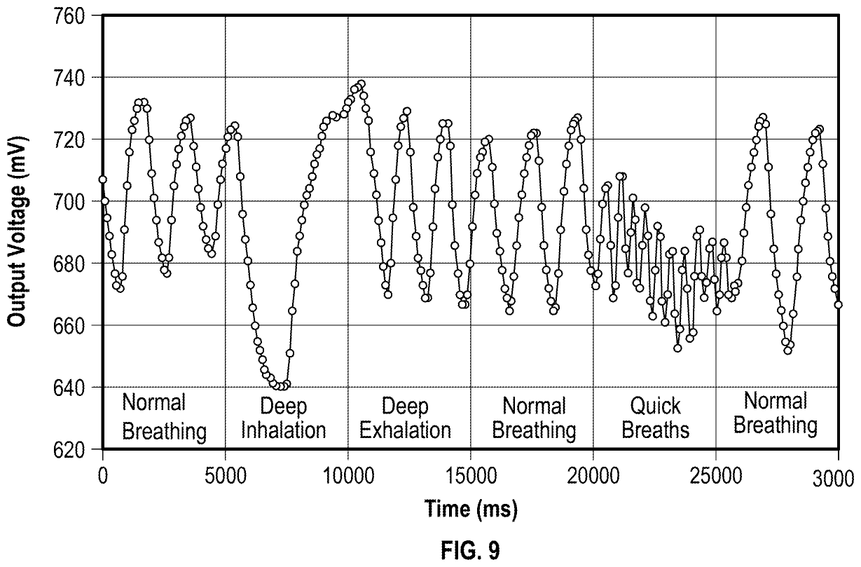

FIG. 9 is an example of a respiratory signal acquired by a strain gauge according to one embodiment of the present disclosure. The respiratory signal can have amplitudes in the millivolt (mV) range. The amplitude and frequency of the respiratory signal can vary across different types of breathing and phases of the respiratory cycle. In one embodiment, inhalation can correspond to a period between a local minimum (trough) and a local maximum (peak) directly following the local minimum. The local minimum can correspond to a maximum deflation of the chest following exhalation, and the following local maximum can correspond to a maximum inflation of the chest following inhalation. In one embodiment, the local minima and maxima can be identified using a peak detection algorithm. The length of an inhalation period can vary based on a wearer's level of physical exertion. For example, in quick breathing, the inhalation period can be approximately 250 milliseconds, while in deep inhalation, the inhalation period can be approximately 2500 milliseconds. The respiratory sensor can record each respiratory phase even during rapid breathing, as illustrated in FIG. 9. The respiratory signal acquired by the sensor can be confirmed to match observed respiration of a wearer.

The audio signal can be collected during one or more respiratory cycles. However, it can be difficult to determine a phase of the respiratory cycle based on the audio signal alone. Vocal cord activity may not be periodic or predictable and can occur during any phase of the respiratory cycle. In one embodiment, the respiratory signal and the audio signal can be synchronized to determine a portion of the audio signal that is acquired during an inhalation period identified in the respiratory signal. In one embodiment, the synchronization of the respiratory signal and the audio signal can be performed during signal acquisition by the microcontroller. For example, a clock of the sound sensor can be tied to a clock of the respiratory sensor so that the sensors share the same clock edges and can acquire data synchronously. In one embodiment, a microcontroller can synchronize the sensors by triggering data acquisition of each of the sensors. For example, the microcontroller can provide a reference clock signal to each of the sensors and/or an acquisition initialization signal. In one embodiment, each sensor can transmit a time-stamped biosignal to the microcontroller. The time stamps can correspond to when the signals were acquired. The time stamps can be generated by a global positioning system (GPS) module or similar timekeeping module that acquires time data from an external source.

In one embodiment, the respiratory signal and the audio signal can be synchronized after signal acquisition. For example, a user device or microcontroller can resample a biosignal so that the biosignals have the same sampling rate. In one embodiment, a sampling period of the sound sensors and the strain gauges can be 10 milliseconds. In one embodiment, a user device or microcontroller can determine an offset (delay) in time between biosignals and can shift one or more biosignals to correct the offset.

In one embodiment, an audio signal can be processed to identify vocal cord activity. In one embodiment, sound caused by vocal cord activity can fall within a frequency range. In one embodiment, sound caused by vocal cord activity can have a frequency greater than a frequency threshold and/or a volume greater than a volume threshold. In one embodiment, the volume threshold can be based on ambient noise or audio that is recorded by the sound sensors when a wearer is at rest or not exerting themself. In one embodiment, the frequency threshold can be approximately 600 Hz, which is higher than the pitch of a normal human voice. In one embodiment, the frequency threshold and/or the volume threshold can be dynamic thresholds. For example, the threshold values for a wearer can be dependent on baseline signals acquired when the wearer is at rest.

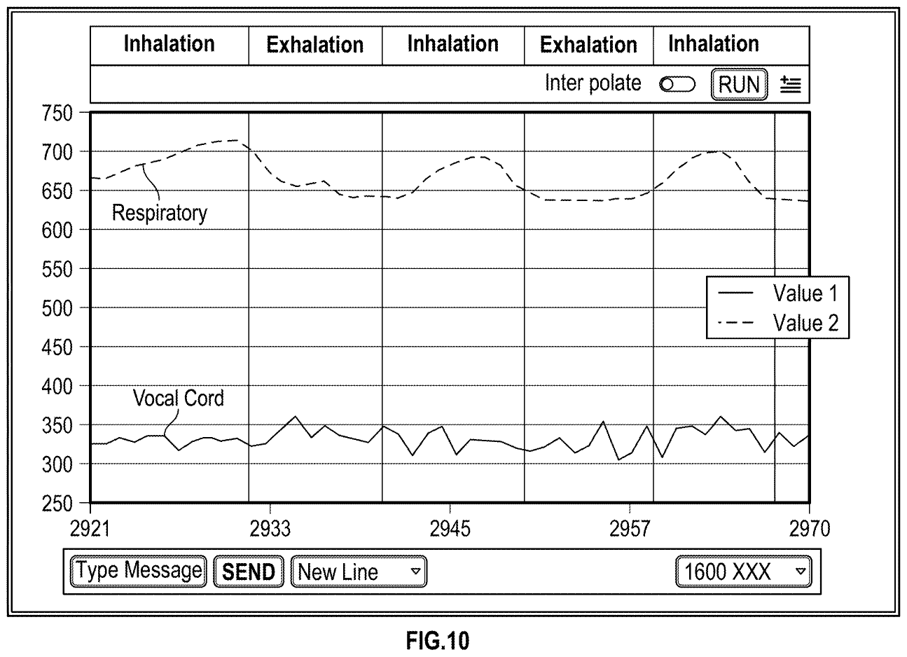

FIG. 10 is an example of a synchronized respiratory signal and audio signal according to one embodiment of the present disclosure. The respiratory signal can be segmented at local minima and maxima to determine periods corresponding to inhalation and exhalation. The audio signal activity during those periods can be used to identify vocal cord activity during inhalation and exhalation. In one embodiment, vocal cord activity can correspond to an audio signature. For example, the frequency (pitch) of the audio signal can be above a frequency threshold during vocal cord activity. In one embodiment, a pattern or level of vocal cord activity can be identified using the synchronized respiratory signal and audio signal. In one embodiment, a certain pattern or level of vocal cord activity can correspond to EILO. For example, the pattern can include a degree of vocal cord activity during inhalation. In one embodiment, the audio signal can indicate movement of supraglottic structures. The movement of supraglottic structures can also correspond to an audio signature.

In one embodiment, vocal cord activity can be identified based on the audio signal using a machine learning method. For example, a machine learning model can be trained on labeled audio signal data corresponding to varying degrees of vocal cord activity. The machine learning model can be trained to extract and identify features of the audio signal data and predict whether the audio signal data is acquired from a patient having EILO based on the extracted features.

In one embodiment, the analysis of the synchronized respiratory signal and audio signal can be used to identify inspiratory stridor and diagnose a patient with EILO. In one embodiment, the user device can generate a diagnosis of EILO based on the synchronized respiratory signal and audio signal. In one embodiment, the user device can determine a probability of EILO. For example, the probability of EILO can be based on a degree and frequency of audio signals corresponding to vocal cord activity during inhalation.

Advantageously, the respiratory signal and the audio signal can be acquired while a patient (wearer) is actively exercising, resulting in biosignal data that is relevant to identifying EILO. The wearable apparatus having respiratory sensors and sound sensors is less invasive than a laryngoscope used to visualize the vocal cords. The wearable apparatus having separate components can also be portable, easy to attach to the body, and convenient. The synchronization of the respiratory signal and the audio signal can also provide accurate data on phases of the respiratory cycle. In this manner, EILO, which affects inhalation, can be distinguished from asthma or other respiratory issues that affect exhalation.

In one embodiment, a wearable apparatus having a sound sensor and a respiratory sensor, as described herein, can be tested and validated in patient trials. The respiratory signals and the audio signals can be accurately synchronized throughout more than an hour of signal acquisition. The wearable apparatus can be secured to the body throughout various forms of exercise, including vertical and horizontal movement. In one embodiment, the wearable apparatus can be secured to wearers of different ages, genders, sizes, etc.

Embodiments of the subject matter and the functional operations described in this specification can be implemented by digital electronic circuitry, in tangibly embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus, such as the electronic device, consumer device, networked electronic device, etc. The computer storage medium can be a machine-readable storage device, a machine-readable storage substrate, a random or serial access memory device, or a combination of one or more of them.

Each of the functions of the described embodiments can be implemented by one or more processing circuits/processing circuitry/processing firmware (may also be referred to as a controller). A processing circuit includes a programmed processor (for example, a CPU of FIG. 13), as a processor includes circuitry. A processing circuit can also include devices such as an application specific integrated circuit (ASIC) and circuit components arranged to perform the recited functions.

The term “data processing apparatus” refers to data processing hardware and may encompass all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA an ASIC.

Computers suitable for the execution of a computer program include, by way of example, general or special purpose microprocessors or both, or any other kind of central processing unit. Generally, a CPU will receive instructions and data from a read-only memory or a random access memory or both. Elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients (user devices) and servers. A client and server are generally remote from each other and typically interact through a communication network. The relationship of client and server arises by virtue of computer programs running on the respective computers and having a client-server relationship to each other. In an embodiment, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

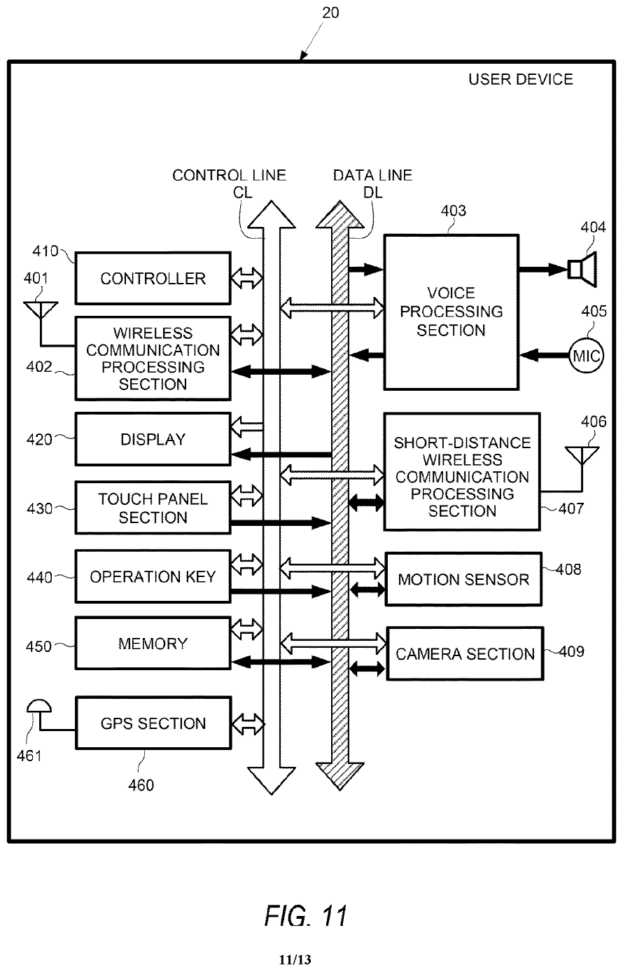

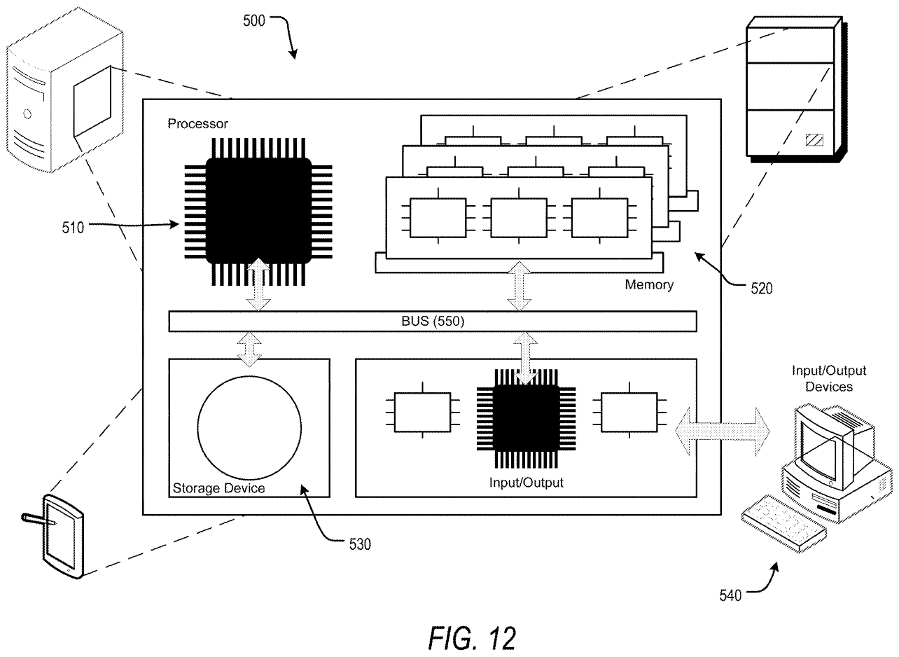

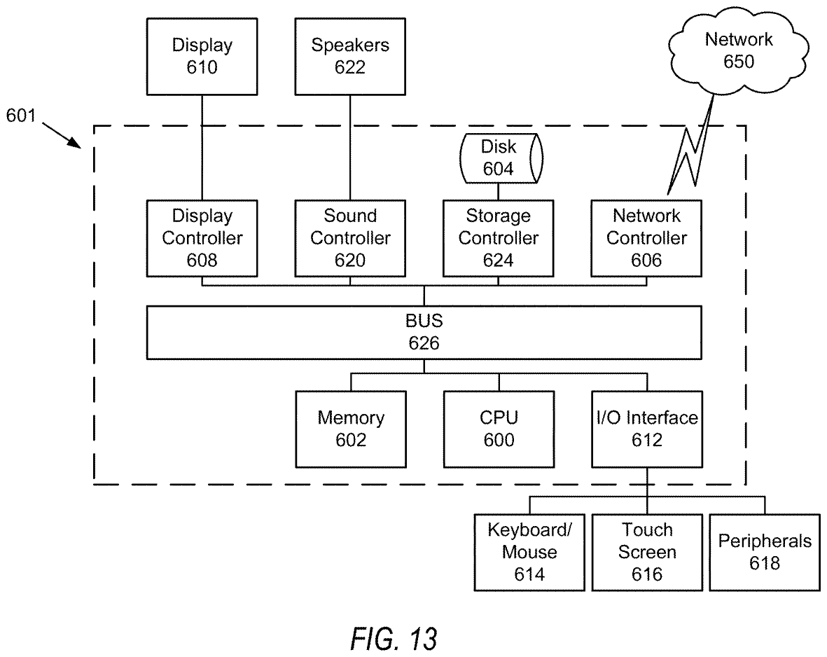

Electronic user device 20 shown in FIG. 11 can be an example of one or more of the devices described herein, including an electronic device configured process biosignals and generate a diagnosis of EILO. In an embodiment, the electronic user device 20 may be a smartphone. However, the skilled artisan will appreciate that the features described herein may be adapted to be implemented on other devices (e.g., a laptop, a tablet, a server, an e-reader, a camera, a navigation device, etc.). In one embodiment, the microprocessor can include components of the user device 20. The user device 20 of FIG. 11 includes processing circuitry, as discussed above. The processing circuitry includes one or more of the elements discussed next with reference to FIG. 11. The electronic user device 20 may include other components not explicitly illustrated in FIG. 11 such as a CPU, GPU, frame buffer, etc. The electronic user device 20 includes a controller 410 and a wireless communication processor 402 connected to an antenna 401. A speaker 404 and a microphone 405 are connected to a voice processor 403.

The controller 410 may include one or more processors/processing circuitry (CPU, GPU, or other circuitry) and may control each element in the user device 20 to perform functions related to communication control, audio signal processing, graphics processing, control for the audio signal processing, still and moving image processing and control, and other kinds of signal processing. The controller 410 may perform these functions by executing instructions stored in a memory 450. Alternatively or in addition to the local storage of the memory 450, the functions may be executed using instructions stored on an external device accessed on a network or on a non-transitory computer readable medium.

The memory 450 includes but is not limited to Read Only Memory (ROM), Random Access Memory (RAM), or a memory array including a combination of volatile and non-volatile memory units. The memory 450 may be utilized as working memory by the controller 410 while executing the processes and algorithms of the present disclosure. Additionally, the memory 450 may be used for long-term storage, e.g., of image data and information related thereto.

The user device 20 includes a control line CL and data line DL as internal communication bus lines. Control data to/from the controller 410 may be transmitted through the control line CL. The data line DL may be used for transmission of voice data, displayed data, etc.

The antenna 401 transmits/receives electromagnetic wave signals between base stations for performing radio-based communication, such as the various forms of cellular telephone communication. The wireless communication processor 402 controls the communication performed between the user device 20 and other external devices via the antenna 401. For example, the wireless communication processor 402 may control communication between base stations for cellular phone communication.

The speaker 404 emits an audio signal corresponding to audio data supplied from the voice processor 403. The microphone 405 detects surrounding audio and converts the detected audio into an audio signal. The audio signal may then be output to the voice processor 403 for further processing. The voice processor 403 demodulates and/or decodes the audio data read from the memory 450 or audio data received by the wireless communication processor 402 and/or a short-distance wireless communication processor 407. Additionally, the voice processor 403 may decode audio signals obtained by the microphone 405.

The user device 20 may also include a display 420, a touch panel 430, an operation key 440, and a short-distance communication processor 407 connected to an antenna 406. The display 420 may be a Liquid Crystal Display (LCD), an organic electroluminescence display panel, or another display screen technology. In addition to displaying still and moving image data, the display 420 may display operational inputs, such as numbers or icons which may be used for control of the user device 20. The display 420 may additionally display a GUI for a user to control aspects of the user device 20 and/or other devices. Further, the display 420 may display characters and images received by the user device 20 and/or stored in the memory 450 or accessed from an external device on a network. For example, the user device 20 may access a network such as the Internet and display text and/or images transmitted from a Web server.

The touch panel 430 may include a physical touch panel display screen and a touch panel driver. The touch panel 430 may include one or more touch sensors for detecting an input operation on an operation surface of the touch panel display screen. The touch panel 430 also detects a touch shape and a touch area. Used herein, the phrase “touch operation” refers to an input operation performed by touching an operation surface of the touch panel display with an instruction object, such as a finger, thumb, or stylus-type instrument. In the case where a stylus or the like is used in a touch operation, the stylus may include a conductive material at least at the tip of the stylus such that the sensors included in the touch panel 430 may detect when the stylus approaches/contacts the operation surface of the touch panel display (similar to the case in which a finger is used for the touch operation).

In certain aspects of the present disclosure, the touch panel 430 may be disposed adjacent to the display 420 (e.g., laminated) or may be formed integrally with the display 420. For simplicity, the present disclosure assumes the touch panel 430 is formed integrally with the display 420 and therefore, examples discussed herein may describe touch operations being performed on the surface of the display 420 rather than the touch panel 430. However, the skilled artisan will appreciate that this is not limiting.

For simplicity, the present disclosure assumes the touch panel 430 is a capacitance-type touch panel technology. However, it should be appreciated that aspects of the present disclosure may easily be applied to other touch panel types (e.g., resistance-type touch panels) with alternate structures. In certain aspects of the present disclosure, the touch panel 430 may include transparent electrode touch sensors arranged in the X-Y direction on the surface of transparent sensor glass.

The touch panel driver may be included in the touch panel 430 for control processing related to the touch panel 430, such as scanning control. For example, the touch panel driver may scan each sensor in an electrostatic capacitance transparent electrode pattern in the X-direction and Y-direction and detect the electrostatic capacitance value of each sensor to determine when a touch operation is performed. The touch panel driver may output a coordinate and corresponding electrostatic capacitance value for each sensor. The touch panel driver may also output a sensor identifier that may be mapped to a coordinate on the touch panel display screen. Additionally, the touch panel driver and touch panel sensors may detect when an instruction object, such as a finger is within a predetermined distance from an operation surface of the touch panel display screen. That is, the instruction object does not necessarily need to directly contact the operation surface of the touch panel display screen for touch sensors to detect the instruction object and perform processing described herein. For example, in an embodiment, the touch panel 430 may detect a position of a user's finger around an edge of the display panel 420 (e.g., gripping a protective case that surrounds the display/touch panel). Signals may be transmitted by the touch panel driver, e.g. in response to a detection of a touch operation, in response to a query from another element based on timed data exchange, etc.

The touch panel 430 and the display 420 may be surrounded by a protective casing, which may also enclose the other elements included in the user device 20. In an embodiment, a position of the user's fingers on the protective casing (but not directly on the surface of the display 420) may be detected by the touch panel 430 sensors. Accordingly, the controller 410 may perform display control processing described herein based on the detected position of the user's fingers gripping the casing. For example, an element in an interface may be moved to a new location within the interface (e.g., closer to one or more of the fingers) based on the detected finger position.

Further, in an embodiment, the controller 410 may be configured to detect which hand is holding the user device 20, based on the detected finger position. For example, the touch panel 430 sensors may detect fingers on the left side of the user device 20 (e.g., on an edge of the display 420 or on the protective casing), and detect a single finger on the right side of the user device 20. In this example, the controller 410 may determine that the user is holding the user device 20 with his/her right hand because the detected grip pattern corresponds to an expected pattern when the user device 20 is held only with the right hand.

The operation key 440 may include one or more buttons or similar external control elements, which may generate an operation signal based on a detected input by the user. In addition to outputs from the touch panel 430, these operation signals may be supplied to the controller 410 for performing related processing and control. In certain aspects of the present disclosure, the processing and/or functions associated with external buttons and the like may be performed by the controller 410 in response to an input operation on the touch panel 430 display screen rather than the external button, key, etc. In this way, external buttons on the user device 20 may be eliminated in lieu of performing inputs via touch operations, thereby improving watertightness.

The antenna 406 may transmit/receive electromagnetic wave signals to/from other external apparatuses, and the short-distance wireless communication processor 407 may control the wireless communication performed between the other external apparatuses. Bluetooth, IEEE 802.11, and near-field communication (NFC) are non-limiting examples of wireless communication protocols that may be used for inter-device communication via the short-distance wireless communication processor 407.

The user device 20 may include a motion sensor 408. The motion sensor 408 may detect features of motion (i.e., one or more movements) of the user device 20. For example, the motion sensor 408 may include an accelerometer to detect acceleration, a gyroscope to detect angular velocity, a geomagnetic sensor to detect direction, a geo-location sensor to detect location, etc., or a combination thereof to detect motion of the user device 20. In an embodiment, the motion sensor 408 may generate a detection signal that includes data representing the detected motion. For example, the motion sensor 408 may determine a number of distinct movements in a motion (e.g., from start of the series of movements to the stop, within a predetermined time interval, etc.), a number of physical shocks on the user device 20 (e.g., a jarring, hitting, etc., of the electronic device), a speed and/or acceleration of the motion (instantaneous and/or temporal), or other motion features. The detected motion features may be included in the generated detection signal. The detection signal may be transmitted, e.g., to the controller 410, whereby further processing may be performed based on data included in the detection signal. The motion sensor 408 can work in conjunction with a Global Positioning System (GPS) section 460. The information of the present position detected by the GPS section 460 is transmitted to the controller 410. An antenna 461 is connected to the GPS section 460 for receiving and transmitting signals to and from a GPS satellite.

The user device 20 may include a camera section 409, which includes a lens and shutter for capturing photographs of the surroundings around the user device 20. In an embodiment, the camera section 409 captures surroundings of an opposite side of the user device 20 from the user. The images of the captured photographs can be displayed on the display panel 420. A memory section saves the captured photographs. The memory section may reside within the camera section 109 or it may be part of the memory 450. The camera section 409 can be a separate feature attached to the user device 20 or it can be a built-in camera feature.

An example of a type of computer is shown in FIG. 12. The computer 500 can be used for the operations described in association with any of the computer-implement methods described previously, according to one implementation. For example, the computer 500 can be an example of an electronic device, such as a computer or mobile device, or a networked device such as a server. The processing circuitry includes one or more of the elements discussed next with reference to FIG. 12. In FIG. 12, the computer 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the computer 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the computer 500. In one implementation, the storage device 530 is a computer-readable medium. In various different implementations, the storage device 530 may be a floppy disk device, a hard disk device, an optical disk device, or a tape device.

The input/output device 540 provides input/output operations for the computer 500. In one implementation, the input/output device 540 includes a keyboard and/or pointing device. In another implementation, the input/output device 540 includes a display unit for displaying graphical user interfaces.

Next, a hardware description of a device 601 according to the present embodiments is described with reference to FIG. 13. In FIG. 13, the device 601, which can be any of the above described devices, including the electronic devices and the networked devices, includes processing circuitry. The processing circuitry includes one or more of the elements discussed next with reference to FIG. 13. The process data and instructions may be stored in memory 602. These processes and instructions may also be stored on a storage medium disk 604 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the device 601 communicates, such as a server or computer.

Further, the claimed advancements may be provided as a utility application, background daemon, or component of an operating system, or combination thereof, executing in conjunction with CPU 600 and an operating system such as Microsoft Windows, UNIX, Solaris, LINUX, Apple MAC-OS and other systems known to those skilled in the art.

The hardware elements in order to achieve the device 601 may be realized by various circuitry elements, known to those skilled in the art. For example, CPU 600 may be a Xenon or Core processor from Intel of America or an Opteron processor from AMD of America, or may be other processor types that would be recognized by one of ordinary skill in the art. Alternatively, the CPU 600 may be implemented on an FPGA, ASIC, PLD or using discrete logic circuits, as one of ordinary skill in the art would recognize. Further, CPU 600 may be implemented as multiple processors cooperatively working in parallel to perform the instructions of the processes described above.

The device 601 in FIG. 13 also includes a network controller 606, such as an Intel Ethernet PRO network interface card from Intel Corporation of America, for interfacing with network 650. and to communicate with the other devices. As can be appreciated, the network 650 can be a public network, such as the Internet, or a private network such as an LAN or WAN network, or any combination thereof and can also include PSTN or ISDN sub-networks. The network 650 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The device 601 further includes a display controller 608, such as a NVIDIA GeForce GTX or Quadro graphics adaptor from NVIDIA Corporation of America for interfacing with display 610, such as an LCD monitor. A general purpose I/O interface 612 interfaces with a keyboard and/or mouse 614 as well as a touch screen panel 616 on or separate from display 610. General purpose I/O interface also connects to a variety of peripherals 618 including printers and scanners.

A sound controller 620 is also provided in the device 601 to interface with speakers/microphone 622 thereby providing sounds and/or music.

The general purpose storage controller 624 connects the storage medium disk 604 with communication bus 626, which may be an ISA, EISA, VESA, PCI, or similar, for interconnecting all of the components of the device 601. A description of the general features and functionality of the display 610, keyboard and/or mouse 614, as well as the display controller 608, storage controller 624, network controller 606, sound controller 620, and general purpose I/O interface 612 is omitted herein for brevity as these features are known.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination.

Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Embodiments of the present disclosure may also be as set forth in the following parentheticals:
(1) A wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry, wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.
(2) The wearable apparatus of (1), wherein the sound sensor is coupled to a neckpiece configured to fit around a neck of the wearer.
(3) The wearable apparatus of (1) to (2), wherein the flexible band is a harness configured to fit around the chest of the wearer.
(4) The wearable apparatus of (1) to (3), wherein the sound sensor is coupled to a wristband.
(5) The wearable apparatus of (1) to (4), wherein the processing circuitry is configured to synchronize the acquisition of the audio signal and the acquisition of the respiratory signal by transmitting an acquisition initialization signal to the sound sensor and the strain gauge.
(6) The wearable apparatus of (1) to (5), wherein the inspiration segment of the respiratory signal is a segment of respiratory signal data between a local minimum and a local maximum directly following the local minimum.
(7) The wearable apparatus of (1) to (6), wherein the processing circuitry is configured to receive the audio signal and the respiratory signal over a wireless communication network.
(8) A wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry.
(9) The wearable apparatus of (8), wherein the sound sensor is coupled to a neckpiece configured to fit around a neck of the wearer.
(10) The wearable apparatus of (8) to (9), wherein the flexible band is a harness configured to fit around the chest of the wearer.
(11) The wearable apparatus of (8) to (10), wherein the sound sensor is coupled to a wristband.
(12) The wearable apparatus of (8) to (11), wherein the processing circuitry is configured to synchronize the acquisition of the audio signal and the acquisition of the respiratory signal by transmitting an acquisition initialization signal to the sound sensor and the strain gauge.
(13) The wearable apparatus of (8) to (12), wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.
(14) The wearable apparatus of (8) to (13), wherein the inspiration segment of the respiratory signal is a segment of respiratory signal data between a local minimum and a local maximum directly following the local minimum.
(15) The wearable apparatus of (8) to (14), wherein the processing circuitry is configured to receive the audio signal and the respiratory signal over a wireless communication network.
(16) A wearable apparatus, comprising: a sound sensor configured to acquire an audio signal from a throat of a wearer; a strain gauge coupled to a flexible band and configured to measure a strain on the strain gauge when the flexible band is in contact with a chest of the wearer during a respiratory cycle of the wearer and output a respiratory signal corresponding to the strain; and processing circuitry contained in a housing coupled to a wristband and configured to synchronize acquisition of the audio signal and acquisition of the respiratory signal, receive the audio signal from the sound sensor and the respiratory signal from the strain gauge, and transmit the audio signal and the respiratory signal to external processing circuitry, wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.
(17) The wearable apparatus of (16), wherein the sound sensor is coupled to a neckpiece configured to fit around a neck of the wearer.
(18) The wearable apparatus of (16) to (17), wherein the flexible band is a harness configured to fit around the chest of the wearer.
(19) The wearable apparatus of (16) to (18), wherein the processing circuitry is configured to synchronize the acquisition of the audio signal and the acquisition of the respiratory signal by transmitting an acquisition initialization signal to the sound sensor and the strain gauge.
(20) The wearable apparatus of (16) to (19), wherein the inspiration segment of the respiratory signal is a segment of respiratory signal data between a local minimum and a local maximum directly following the local minimum.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A wearable apparatus, comprising:
a sound sensor configured to acquire an audio signal from a throat of a wearer;
a strain gauge coupled to a flexible band and configured to measure a strain on the strain gauge when the flexible band is in contact with a chest of the wearer during a respiratory cycle of the wearer and output a respiratory signal corresponding to the strain; and
processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry,
wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.

2. The wearable apparatus of claim 1, wherein the sound sensor is coupled to a neckpiece configured to fit around a neck of the wearer.

3. The wearable apparatus of claim 1, wherein the flexible band is a harness configured to fit around the chest of the wearer.

4. The wearable apparatus of claim 1, wherein the sound sensor is coupled to a wristband.

5. The wearable apparatus of claim 1, wherein the processing circuitry is configured to synchronize the acquisition of the audio signal and the acquisition of the respiratory signal by transmitting an acquisition initialization signal to the sound sensor and the strain gauge.

6. The wearable apparatus of claim 1, wherein the inspiration segment of the respiratory signal is a segment of respiratory signal data between a local minimum and a local maximum directly following the local minimum.

7. The wearable apparatus of claim 1, wherein the processing circuitry is configured to receive the audio signal and the respiratory signal over a wireless communication network.

8. A wearable apparatus, comprising:
a sound sensor configured to acquire an audio signal from a throat of a wearer;
a strain gauge coupled to a flexible band and configured to measure a strain on the strain gauge when the flexible band is in contact with a chest of the wearer during a respiratory cycle of the wearer and output a respiratory signal corresponding to the strain; and
processing circuitry contained in a housing and configured to synchronize acquisition of the audio signal and acquisition of the respiratory signal, receive the audio signal from the sound sensor and the respiratory signal from the strain gauge, and transmit the audio signal and the respiratory signal to external processing circuitry.

9. The wearable apparatus of claim 8, wherein the sound sensor is coupled to a neckpiece configured to fit around a neck of the wearer.

10. The wearable apparatus of claim 8, wherein the flexible band is a harness configured to fit around the chest of the wearer.

11. The wearable apparatus of claim 8, wherein the sound sensor is coupled to a wristband.

12. The wearable apparatus of claim 8, wherein the processing circuitry is configured to synchronize the acquisition of the audio signal and the acquisition of the respiratory signal by transmitting an acquisition initialization signal to the sound sensor and the strain gauge.

13. The wearable apparatus of claim 8, wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.

14. The wearable apparatus of claim 13, wherein the inspiration segment of the respiratory signal is a segment of respiratory signal data between a local minimum and a local maximum directly following the local minimum.

15. The wearable apparatus of claim 8, wherein the processing circuitry is configured to receive the audio signal and the respiratory signal over a wireless communication network.

16. A wearable apparatus, comprising:
a sound sensor configured to acquire an audio signal from a throat of a wearer;
a strain gauge coupled to a flexible band and configured to measure a strain on the strain gauge when the flexible band is in contact with a chest of the wearer during a respiratory cycle of the wearer and output a respiratory signal corresponding to the strain; and
processing circuitry contained in a housing coupled to a wristband and configured to synchronize acquisition of the audio signal and acquisition of the respiratory signal, receive the audio signal from the sound sensor and the respiratory signal from the strain gauge, and transmit the audio signal and the respiratory signal to external processing circuitry,
wherein the external processing circuitry is configured to identify an inspiration segment of the respiratory signal corresponding to an inspiration phase of the respiratory cycle and detect inspiratory stridor based on a segment of the audio signal acquired during acquisition of the inspiration segment of the respiratory signal.

17. The wearable apparatus of claim 16, wherein the sound sensor is coupled to a neckpiece configured to fit around a neck of the wearer.

18. The wearable apparatus of claim 16, wherein the flexible band is a harness configured to fit around the chest of the wearer.

19. The wearable apparatus of claim 16, wherein the processing circuitry is configured to synchronize the acquisition of the audio signal and the acquisition of the respiratory signal by transmitting an acquisition initialization signal to the sound sensor and the strain gauge.

20. The wearable apparatus of claim 16, wherein the inspiration segment of the respiratory signal is a segment of respiratory signal data between a local minimum and a local maximum directly following the local minimum.
